(19) United States
(12) Patent Application Publication
(10) Pub. No.: US 2004/0180104 A1
(43) Pub. Date: Sep. 16, 2004

(54) Herbal composition for preventing and/or treating conditions in associated with depression

(76) Inventor: Chih Hui Lin, Taichung (TW)

Correspondence Address: HARNESS, DICKEY & PIERCE, P.L.C., P.O. BOX 8910, RESTON, VA, 20195, US

(21) Appl. No.: 10/384,544

(22) Filed: Mar. 11, 2003

(51) Int. Cl. A61K 35/78

(52) U.S. Cl. 424/735; 424/746; 424/750; 424/757; 424/725; 424/756; 424/736

(57) ABSTRACT

The present invention relates to a new herbal composition for the prevention and/or treatment of the conditions in associated with depression. Also provided is a method of treating and/or preventing the conditions associated with depression by administering the herbal composition of the invention.

### BACKGROUND OF THE INVENTION

1. Field of the Invention

The present invention relates to a new herbal composition for the prevention and/or treatment of the conditions in associated with depression.

2. Description of the Prior Art

Major depression is characterized by feelings of intense sadness and despair, mental slowing and loss of concentration, pessimistic worry, agitation, and self-deprecation. Physical changes also occur, especially in severe or “melancholic” depression. These include insomnia or hypersomnia, anorexia and weight loss (or sometimes overeating), decreased energy and libido, and disruption of normal circadian rhythms of activity, body temperature, and many endocrine functions. There are multiple causes of depression and the causes may include genetic, familial, biochemical, physical, social and psychological factors. Major depression occurs in all groups of people and affects both sexes, with an increased incidence in women, most common in adolescent girls, affecting 5%-10% of the population.

Depressive disorders are frequently managed with long-term use of antidepressant medication. The following antidepressant drugs are widely used in the world: selective sertonin-reuptake inhibitors (SSRIs), tricyclic antidepressants (TCAs) and monoamine oxidase inhibitors (MAOIs). A number of SSRIs such as setraline, fluoxetine, paroxetine, and fluvoxamine, TCAs such as ELAVIL™, MAOIs such as Nardil, Parnate, and L-deprenyl were developed to treat the depression. However, these drugs are potent, often generating problematic side effects such as lethargy, clouded thinking, lack of ability to concentrate, and sexual dysfunction. For example, the side effects of using TCAs are sedation and weight gain and the MAOIs cause dietary and drug interactions. In particular, sexual dysfunction has long been noted as both a symptom of depressive illness and a side effect of many antidepressants. Ferguson J. M. indicated that most antidepressants interfere with human sexual functioning (Ferguson J M. J Clin Psychiatry 2001; 62 Suppl 3:22-34). Moreover, some new antidepressants with reduced side effects such as mirtazapine, bupropion and nefazodone were further developed. However, these antidepressants which are administered in a long-term will cause dry mouth and oral infection.

A herb St. John's wort has been found to improve depression conditions and has a reduced side effect (Ghosal and Shibnath, U.S. Pat. No. 6,224,906). Therefore, the researchers gradually attach importance to the development of the herb drugs. However, the St. John's wort does not have strong efficacy in the treatment of depressant, which only can be used in mild to moderate depression. It is continually desirable to provide herbal drugs which have improved anti-depression properties.

### SUMMARY OF THE INVENTION

The present invention relates to a herbal composition, comprising a herb for promoting blood circulation and reducing Congealed Blood or an extract thereof, a herb for transquility or an extract thereof, a herb for sedative or an extract thereof, and a herb for the flow of qi or an extract thereof; wherein the herb for promoting blood circulation and reducing Congealed Blood is selected from the group consisting of Salvia multiorhiza, Lablab pruprueus, Dendranthema×grandiflorum, Cyperus rotundus, Prunus persica, Prunus davidiana, Paeonia suffruticosa, Senna tora, Tribulus terrestris, Paeonia lactiflora, Crocus sativus, Andrographis paniculata, Ligusticum chuanxiong, Gardenia jasminoides, and Cryptotaenia japonica, the herb for transquility is selected from the group consisting of Lilium brownii, Ziziphus jujuba, Polygala tenuifolia Atractylodes macrocephala, Triticum aestivum, Acorus calamus, Anemarrhena asphodeloides and Phyllostachys nigra, and Ocimum basilicum and the herb for sedative is selected from the group consisting of Rehmannia glutinosa, Gentiana triflora, Gentiann scabra and Schisandra chinensis and the herb for the flow of qi is selected from the group consisting of Citrus sinensis, Citrus aurantium, Citrus reticulata, Perilla frutescens, Centella asiatica, Ligusticum sinense, Angelica sinensis, Curcuma longa, Glycyrrhiza uralensis, Lindera strychnifolia, Gastrodia elata, Saussurea costus, Sparganium stoloniferum, Foeniculum vulgare, Angelica pubescens, Arisaema amurense, Acorus gramineus, Anemone altaica, Gentiana macrophylla, Gentiana dahurica.

The invention also relates to a method of treating and/or preventing the conditions associated with depression, comprising administering to a subject in need thereof an effective amount of the composition of the invention.

### DETAILED DESCRIPTION OF THE INVENTION

The present invention relates to a novel herbal composition that provides anti-depression effects. The herbal composition of the invention has improved efficacy in preventing and/or treating the conditions in associated with depression and does not cause any side effects.

### Herbal Composition

The present invention provides a herbal composition, comprising a herb for promoting blood circulation and reducing Congealed Blood or an extract thereof, a herb for transquility or an extract thereof, a herb for sedative or an extract thereof, and a herb for flow of qi or an extract thereof; wherein the herb for promoting blood circulation and reducing Congealed Blood is selected from the group consisting of Salvia multiorhiza, Lablab pruprueus, Dendranthema×grandiflorum, Cyperus rotundus, Prunus persica, Prunus davidiana, Paeonia suffruticosa, Senna tora, Tribulus terrestris, Paeonia lactiflora, Crocus sativus, Andrographis paniculata, Ligusticum chuanxiong, Gardenia jasminoides, and Cryptotaenia japonica, the herb for transquility is selected from the group consisting of Lilium brownii, Ziziphus jujuba, Polygala tenuifolia Atractylodes macrocephala, Triticum aestivum, Acorus calamus, Anemarrhena asphodeloides and Phyllostachys nigra, and Ocimum basilicum and the herb for sedative is selected from the group consisting of Rehmannia glutinosa, Gentiana triflora, Gentiann scabra and Schisandra chinensis and the herb for the flow of qi is selected from the group consisting of Citrus sinensis, Citrus aurantium, Citrus reticulata, Perilla frutescens, Centella asiatica, Ligusticum sinense, Angelica sinensis, Curcuma longa, Glycyrrhiza uralensis, Lindera strychnifolia, Gastrodia elata, Saussurea costus, Sparganium stoloniferum, Foeniculum vulgare, Angelica pubescens, Arisaema amurense, Acorus gramineus, Anemone altaica, Gentiana macrophylla, Gentiana dahurica. Preferably, the herb for promoting blood circulation and reducing Congealed Blood is selected from the group consisting of Salvia multiorhiza and Andrographis paniculata, the herb for transquility is selected from the group consisting of Lilium brownie and Polygala tenuifolia, the herb for sedative is selected from the group consisting of Rehmannia glutinosa and Schisandra chinensis, and the herb for the flow of qi is selected from the group consisting of Citrus sinensis, Citrus aurantium, Citrus reticulata and Perilla frutescens. More preferably, the herb for promoting blood circulation and reducing Congealed Blood is Salvia multiorhiza, the herb for transquility is Lilium brownie, the herb for sedative is Rehmannia glutinosa, and the herb for the flow of qi is Citrus sinensis.

Chinese medicine is a complete system of medicine encompassing the entire range of human experience. Chinese medicine is a combination of poetry and science based on a dynamic understanding of energy and flow. According to traditional Chinese medical theory, the fundamental substances of the body are qi (pronounced “chee”), blood, jing, shen, and body fluids. The proper formation, maintenance, and circulation of these substances are essential to health. As used herein, the concept of qi is based on the ancient Chinese initial understanding of natural phenomena. That is, qi is the most basic substance by which the world is comprised. Everything in the universe results from the movements and changes of qi. The qi in the human body is different in classification and formation. Generally speaking, the qi has no more than two sources. One is the innate vital substance—one inherits from one's parents from birth. The other is the food essence and fresh air—one receives from air, water and food in the natural world. The meaning of qi has two aspects: one refers to the vital substance comprising the human body and maintaining its life activities, such as qi of water and food (food essence), qi of breathing (breathing nutrients) and so on. The other refers to the physiological function of viscera and bowels, channels, and collaterals, such qi of the heart, the lung, the spleen and the stomach and so on. The deficiency of qi is tiredness and underfunction: some part or parts of the body fail to do their jobs. The usual signs of the deficiency include a low or weak voice that runs out of breath easily, sweating when there is no obvious reason for it, tiredness, runny stools, and lack of appetite. There are a lot of more specific signs of qi deficiency. For example, if lung qi isn't working properly, we will be short of breath, pale, weak voiced, and often catch colds. If Stomach qi under-performs, we will lose our appetite, cannot taste food properly, feel full too quickly when we eat, get runny or loose stools, and feel weak especially in the morning. Both of these qi deficiencies can lead to general tiredness. When the Heart is under qi deficiency, we will get palpitations too: each of the Zang-fu has its own picture when there is a deficient supply of qi.

As used herein, “Blood” is actually another form of qi, just thicker than the ‘qi-stuff’ that gives us vitality and life. Blood is the mother energy in the body: it makes muscles, bones, brain, tissues and all of the rest. It provides a resting place for the qi. Indeed, the Chinese say that whereas Qi leads the Blood, Blood is the mother of qi. So they use the term Blood in a different way from Western Medicine. From the Chinese point of view, what we call blood is just a red liquid, unless it has qi and becomes Blood. How does the red stuff become Blood? The beginning of the process depends on good Stomach qi to feel hungry. By eating, swallowing and digesting the right food and liquids, the Stomach qi can transform them into the red stuff which is the first stage of Blood. This ascends to the Lungs, which move it towards the Heart, giving it energy. But it is only in the Heart that it becomes Blood. Hence, the saying in Traditional Chinese Medicine (TCM) is that the ‘Heart governs Blood’. The main function of the Blood is to nourish the body, to moisten it and to provide a place for the Mind to dwell and thrive. Words that describe the actions of Blood include stability, suppleness, good texture, resourcefulness, and good complexion. There are four kinds of Blood disharmony. When Blood-deficient, we get pallor, dryness, cracking of joints or nails, uneasiness, irritability, restlessness, poor memory and concentration, falling hair, depression, insomnia, poor sight or tired eyes, impotence or scanty periods and infertility, not to mention dizziness and numbness. (Not necessarily all together. However, some of these problems can occur from other causes in TCM too.) And that is only for starters. Blood deficiency occurs when the Spleen energy is deficient, meaning that our digestion fails to ingest the right food and turns it into blood.

Chinese medicine normally contains several herbs. One commonly used format or template for designing herbal formulas is based on the monarchical form of government. At the top is the king or emperor. Next are the ministers or deputies. Last are the assistants or adjutants. Any herb can fill any of these roles. Which role depends on which herbal formula the herb is used in. These roles work together in the following ways: (i) King herb—primarily responsible for dealing with the syndrome under treatment; (ii) Subject herb—assist the king herb by reinforcing the action of the king herb and treating the concurrent syndrome; (iii) Assistant herb—play one of the three roles: assist the king herb, control the undesirable actions of the king herb, or reduce the toxic effects; and (iv) Servant herb: play two basic roles: direct the formula to the affected region, and harmonize the herbs in the formula.

The above-mentioned Chinese medicine theory can be found at the website: www.tcm.health-info.org, www.chinesemedicinesampler.com, www.acuhealing.com, http://website.lineone.net/˜balloonz/enhc.htm etc. The herbal composition of the invention is developed based on the above-mentioned Chinese medicine theory.

In addition, the information regarding the herbs as described herein can be found at the website: www.ibiblio.org/pfaf.

Herbs for Promoting Blood Circulation and Reducing Congealed Blood

According to the invention, the herb for promoting blood circulation and reducing Congealed Blood used in the composition of the invention plays the role of the king herb which is mainly responsible for the anti-depression effect. According to the invention, the herb for promoting blood circulation and reducing Congealed Blood is selected from the group consisting of Salvia multiorhiza or the extract thereof, Lablab pruprueus or the extract thereof, Dendranthema×grandiflorum or the extract thereof, Cyperus rotundus or the extract thereof, Prunus persica or the extract thereof, Prunus davidiana or the extract thereof, Paeonia suffruticosa or the extract thereof, Senna tora or the extract thereof, Tribulus terrestris or the extract thereof, Paeonia lactiflora or the extract thereof, Crocus sativus or the extract thereof, Andrographis paniculata or the extract thereof, Ligusticum chuanxiong or the extract thereof, Gardenia jasminoides or the extract thereof, and Cryptotaenia japonica or the extract thereof. According to the invention, the above-mentioned herbs have the efficacy in promoting blood circulation and reducing Congealed Blood. The above herbs for promoting blood circulation and reducing Congealed Blood are capable of increasing blood flow and improving circulatory problems. The Latin name, synonyms, possible synonyms and common names of the above herbs for promoting blood circulation and reducing Congealed Blood are shown in below Table 1:

TABLE 1

| Latin name | Synonyms | Possible synonyms | Common names |
|---|---|---|---|
| Salvia multiorrhiza | | | Dan Shen |
| Lablab pruprueus | Lablab vulgaris, | Dolichos purpureus, | E Mei Tou, Huzi-Mame, |
| | Lablab niger, | L. leucocarpos, | Hyacinth Bean, |
| | Dolichos lablab | L. nigra, | Hyacinth-bean, |
| | | L. purpureus subsp. | Hyacinthbean, |
| | | Purpurfeus | Kachang Kara, Kara-kara, |
| | | | Katchang Bado, Kekara, |
| | | | Lablab, Pien Tou, |
| | | | Val Dal, Yen Li Tou |
| Dendranthema x grandiflorum | Dendranthema x | Anthemis grandiflorum, | Chii Hua, Chu Hua, |
| | morifolium, | Anthemis stipulacea, | Chu Hua Chiu, |
| | Chrysanthemum x | Chrysanthemum indicum x | Florist's Chrysanthemum |
| | morifolium, | sinense, | |
| | Chrysanthemum sinense | Chrysanthemum morifolium, | |
| | | Chrysanthemum | |
| | | stipulaceum, | |
| | | D. morifolium, | |
| | | Matricaria morifolia | |
| Cyperus rotundus | Pycreus rotundus, | C. hexastachys | Boeai, Ching San Leng, |
| | Cyperus hexastachyos | | Galingale, Hama-Suge, |
| | | | Hsiang Fu, Mootha, |
| | | | Hsiang Fu Tzu, Mota, |
| | | | Muskezamin, Musta, |
| | | | Mustaka, Mutha, Nut grass, |
| | | | Purple Flat Sedge, |
| | | | Rockoet Teki, |
| | | | So Ken Chiu, So Ts'Ao, |
| | | | Souchet, Tage-tage, |
| | | | Teki, Topalak, Woeta |
| Prunus persica | Persica vulgaris, | Amygdalus persica, | Khokh, Melocotonero, |
| | Amygdalis persicus | Amygdalus petrsica var. | Momo, Peach, |
| | | camelliiflora, | Peach Tree, Pecher, |
| | | Amygdalus persica var. | Pesco, Seftali Agaci, |
| | | densa, | Sheftali, T'Ao P'I Chiu, |
| | | P. persica var. camelliiflora, | Tao Ren |
| | | P. persica var. densa, | |
| | | P. persica var. persica, | |
| | | Persica platycarpa | |
| Prunus davidiana | Persica davidiana, | | |
| | Amygdalus davidiana | | |
| Paeonia suffruticosa | Paeonic moutan | P. arborea | Botan, Dang Pi, |
| | | | Hua Wang, Mou Tan, |
| | | | Pai Liang Chin, |
| | | | Tan Hsu, Tan Ken, |
| | | | Tree Peony |
| Senna tora | Cassia tora | Cassia obtusifolia, | Brusca Cimarrona, Cassia, |
| | | Cassia tora auct. non, | Foetid, Chinese Senna, |
| | | Emelista tora, | Coffeeweed, |
| | | Emelista tora sensu, | Gelenggang Kechil, |
| | | S. obtusifolia | Gelenggang Padang, |
| | | | Jue-ming-zi, |
| | | | Penitora, Pistache Marron, |
| | | | Senna, Sickle Senna, |
| | | | Sickle Wild Sensitive-plant, |
| | | | Ts'Ao Chueh Ming |
| Tribulus terrestris | Gokshura | | Gokharu, Nerinci, |
| | | | Calthrops |
| Paeonia lactiflora | Paeonia edulis, | P. officinalis Thunb. | Bai Shao, Nd, |
| | Paeonia albiflora | | Chin Shao Yao, |
| | | | Chinese Peony, Nd, Shao |
| | | | Yao, |
| | | | Syakuyaku |
| Crocus sativus | | | Asian Saffron, Azafran, |
| | | | Bulgarian Saffron, Crocus, |
| | | | Fan Hung Hua, |
| | | | Greek Saffron, |
| | | | Italian Saffron, |
| | | | Koema-koema, Kumkum, |
| | | | Persian Saffron, |
| | | | Po Fu Lan, Sa Fa Ang, |
| | | | Sa'Faran, Saffron, |
| | | | Saffron Crocus, Safran, |
| | | | Sahuran |
| Andrographis paniculata | | | |
| Ligusticum chuanxiong | | L. chinensis, ligustum | Chinese Privet, Kau-pau |
| | | indicum, Ligustrum | |
| | | microcarpum, Ligustrum | |
| | | sinense, Ligustrum sinense | |
| | | var. stauntonii, Ligustrum | |
| | | stauntonii, Ligustrum | |
| | | villosum, Phillyrea indica | |
| Gardenia jasminoides | | | |

Salvia multiorhiza is tending to grow to 0.6 m, which has long been used in Chinese medicine. The recent research showed that it can be used in heart and circulatory problems (Chevallier. A., The Encyclopedia of Medicinal Plants Dorling Kindersley, London 1996, ISBN 9-780751-303148.) According to the Chinese medicine theory, the salvia miltiorrhiza is used in the treatment of evil qi in the heart and abdomen, continual gurgling of the intestines like water running, cold and heat, and gatherings and accumulations. (Chevallier. A. The Encyclopedia of Medicinal Plants Dorling Kindersley. London 1996 ISBN 9-780751-303148).

Lablab purpureus is tending to grow to 2 m. Lablab purpureus is anticholesterolemic, antidote, antivinous, carminative and hypoglycaemic and can be historically used in the treatment of cholera, vomiting, diarrhea, leucorrhoea, gonorrhoea, alcoholic intoxication and globefish poisoning (A Barefoot Doctors Manual. Running Press 1975 ISBN 012-126540-x.)

Dendranthema×grandiflorum is tending to grow to 1.5 m. Dendranthema×grandiflorum has antibacterical, antifungal, anti-inflammatory, carminative, depurative, febrifuge, ophthalmic, refrigerant and sedative effects and can be used in the treatment of hypertension, coronary heart diseases and angina (Bown. D., Encyclopaedia of Herbs and their Uses, Dorling Kindersley, London. 1995, ISBN 0-7513-020-31.)

Cyperus rotundus is tending to grow to 0.6 m. Historically, it can be used in the treatment of digestive problems and menstrual complaints (Bown. D., Encyclopaedia of Herbs and their Uses, Dorling Kindersley, London. 1995, ISBN 0-7513-020-31.)

Prunus persica is tending to grow to 6 m. Historically, it can be used in the treatment of gastritis, whooping cough, coughs and bronchitis (Bown. D., Encyclopaedia of Herbs and their Uses, Dorling Kindersley, London. 1995, ISBN 0-7513-020-31.)

Prunus davidiana is tending to grow to 9 m. Historically, it can be used in the treatment of amenorrhoea, dysmenorrhoea, traumatic pain due to blood stasis, constipation of aged and debilitated patients, coughs and asthma (Yeung Him-Che, Handbook of Chinese Herbs and Formulas, Institute of Chinese Medicine, Los Sangeles, 1985.)

Paeonia suffruticosa is tending to grow to 2 m. Historically, it can be used in the treatment of fevers, boils, menstrual disorders, nosebleeds, ulcers, irritability and gastro-intestinal infections (Bown. D., Encyclopaedia of Herbs and their Uses, Dorling Kindersley, London. 1995, ISBN: 0-7513-020-31.)

Senna tora prefers light, medium and heavy soils. Historically, it can be used in the treatment of skin diseases.

Tribulus terrestris is an outstanding remedy in urogenital disease, promoting urine flow, soothing the mucosa, and aiding in the excretion of stones and calculi (Frawley et al., 1986, The Yoga of Herbs: An Ayurvedic Guide to Herbal Medicine. Santa Fe: Lotus Press.) In traditional Chinese medicine, Tribulus terrestris is used in the treatment of headache, vertigo and dizziness due to ascentant Liver yang, and dispels Wind-heat for red eyes and excessive tearing, promotes lactation, and is used in the treatment of hives.

Paeonia lactiflora is tending to grow to 0.75 m. Historically, it can be used in the treatment of haemorrhoids and varicose veins (Page M., The Gardener's Guide to Growing Peonies, Newton Abbot, 1997, ISBN 07153 0531 X.)

Crocus sativus is tending to grow to 0.1 m. Historically, it can be used in the treatment of chronic haemorrhages in the uterus and period pains and the induction of menstruation.

Andrographis paniculata has been widely used in traditional Chinese medicine as an antiflammatory and antipyretic drug for the treatment of cold, fever, laryngitis and diarrhea.

Ligusticum sinense is tending to grow to 1 m. Historically, it can be used in the treatment of menstrual disorders, postpartum bleeding, coronary heart disease, poor circulation and headaches (Bown. D., Encyclopaedia of Herbs and their Uses, Dorling Kindersley, London. 1995, ISBN 0-7513-020-31.)

Herb for Transquility

According to the invention, the herb for transquility used in the composition of the invention plays the role of the Subject herb, which reinforces the effect of the herb of promoting blood circulation and reducing Congealed Blood. According to the invention, the herb for transquility is selected from the group consisting of Lilium brownie or the extract thereof, Ziziphus jujube or the extract thereof, Polygala tenuifolia or the extract thereof, Atractylodes macrocephala or the extract thereof, Triticum aestivum or the extract thereof, Acorus calamus or the extract thereof, Anemarrhena asphodeloides or the extract thereof and Phyllostachys nigra or the extract thereof. The above herbs for transquility have sedative effect and are capable of relieving anxiety and depression. The Latin name, synonyms, possible synonyms and common names of the above-mentioned herbs for transquility are shown in below Table 2:

TABLE 2

| Latin name | Synonyms | Possible synonyms | Common names |
|---|---|---|---|
| Lilium brownie | Lilium odorum | | Azucena, Bayaz Zanbag, |
| | Lilium candidum | | Beyaz Zambak, Chia Pai |
| | | | Ho, Hsien Pai Ho, Lily, |
| | | | Madonna, Lily White, Lirio |
| | | | De Jardin, Lis, Madonna |
| | | | Lily, Pai Ho, Pai Ho Kan, |
| | | | Sawsan Abyadh, Yeh Pai |
| | | | Ho, Zanbaq, |
| Ziziphus jujube | Ziziphus zizyphus, | Rhamus jujuba, Rhamus | Azufaifo, Bedara China, |
| | Ziziphus vulgaris, | zizyhus, Z. jujube, Z. | Chinese Date, Chinese |
| | Ziziphus sativa | mauritiana, Z. spinosa, Z. | Jujube, Common Jujube, |
| | | lotos, Z. sativa, | Dara, Hong Zao, Indian |
| | | | Jujube, Jujube, Jujubier, |
| | | | Kan Tsao, Kola, Liane |
| | | | Crocs Chien, Liang Tsao, |
| | | | Mei Tsao, Nabug, Nan |
| | | | Tsao, Pel Tsao, Perita |
| | | | haitiana, Petite Pomme, |
| | | | Pomme Malcadi, Ponsere, |
| | | | Suan Tsao, Ta Tsao, Tsao, |
| | | | Unnab, Unnap Agaci, |
| | | | Widara, |
| Atractylodes macrocephala | | | |
| Polygala tenuifolia | | | |
| Platycodon grandiflorus | Campanula grandiflora, | P. glaucus, | Balloon Flower, |
| | Campanula glauca | P. grandiflorum | Ballon-flower, Baloon |
| | | | Flower, Blue Balloon |
| | | | Flower, Chieh K'Eng, |
| | | | Chieh Keng, Chinese |
| | | | Bellflower, K'U Chieh |
| | | | Keng, Kikyoo, Lu T'Ou. |
| Acorus calamus | | A. americanus, | Acore Vrai, Acoro |
| | | A. calamus auct. non, | Aromatico, Acorus, Agri |
| | | A. calamus var. | Turke, Azakegeri, Bach, |
| | | americanus, | Calamo Aromatico, |
| | | A. calmus. | Calamus, Calmus, Calomo |
| | | | Aromatico, |
| | | | Ch'AngP'UChiu, Cinnamon |
| | | | Sedge, Djerango, Doringo, |
| | | | Ganoeak, gladdon, |
| | | | Jariangau, Jerangau, |
| | | | Jeringau, Kaliraga, |
| | | | Kalmoes, Kalmos, Kalmus, |
| | | | Myrtle Grass, Myrtle |
| | | | Sedge, Sarango, Shui |
| | | | Ch'Ang Pu, Sweet cane, |
| | | | Sweet Flag, Sweet Flagg, |
| | | | Sweet Myrtle, Sweet Root, |
| | | | Sweet Rush, Sweet Sedge, |
| | | | Sweet-flag, Sweetflag, |
| | | | Cacha, Vaj |
| Triticum aestivum | Triticum vulgare, | T. aestivum subsp. | Bread Wheat, Bugday, |
| | Triticum sativum, | Aestivocompactum, | Cheng Ping, Common |
| | | T. aestivum subsp. Aestivum, | Wheat, Fou Mai, Frumint, |
| | | T. aestivum var. graecum, | Hsiao Mai, Hui Mien, Ka |
| | | T. aestivum var. muticum, | Shih Tso, Lai, Mai Ch'Ao, |
| | | T. hybernum, | Mai Fu, Mai Fu Tzu, Man |
| | | T. macha, | Tou, Mien, Mien Chin, |
| | | T. muticum, | Mien Fen, Mo Mo, Pai |
| | | T. sphaerococcum, | Mien, Tarwe, Trigo, Wheat |
| Anemarrhena asphodeloides | | A. asphodelioides | Chih Mu, Huo Mu, Lien |
| | | | Mu, Ti Shen |
| Phyllostachys nigra | Bambusa nigra | P. henionis, | Black bamboo |
| | | P. nigra f. boryana, | |
| | | P. nigra f. muchisasa, | |
| | | P. nigra f. punctata, | |
| | | P. nigra var. henionis, | |
| | | P. nigra var. punctata, | |
| | | P. puberula f. | |
| | | nigro-punctata, | |
| | | P. puberula var. boryana, | |
| | | P. puberula var. muchisasa, | |
| | | P. puberula var. nigra, | |

Lilium brownii is tending to grow to 1.2 m. Historically, it can be used in the treatment of coughs and haematemesis due to deficiency condition, anxiety, apprehension, cedema and difficult urination.

Ziziphus jujube is tending to grow to 10 m. Historically, it can be used in aiding weight gain, improving muscular strength and increasing stamina. In Chinese medicine, it is prescribed as a tonic to strengthen liver function. (Chevallier, A. The Encyclopedia of Medicinal Plants Dorling Kindersley. London 1996 ISBN 9-780751-303148.)

Polygala tenuifolia is tending to grow to 0.2 m. Historically, it can be used in the treatment of coughs with profuse phlegm, bronchitis, insomnia, palpitations, poor memory, anxiety, depression and nervous tension. (Bown. D., Encyclopaedia of Herbs and their Uses, Dorling Kindersley, London. 1995, ISBN 0-7513-020-31.)

Atractylodes macrocephala is tending to grow to 0.5 m, which is widely used in traditional Chinese medicine. Historically, it can be used in the treatment of poor appetite, dyspepsia, abdominal distension, chronic diarrhea, oedema and apontaneous sweating. (Yeung Him-Che, Handbook of Chinese Herbs and Formulas, Institute of Chinese Medicine, Los Angeles, 1985.)

Triticum aestivum is tending to grow to 1.5 m. Historically, it can be used in the treatment of night sweats and spontaneous sweating. (Yeung Him-Che, Handbook of Chinese Herbs and Formulas, Institute of Chinese Medicine, Los Sangeles, 1985.)

Acorus calamus is tending to grow to 1 m. Historically, it can be used in the treatment of digestive complaints, bronchitis, sinusitis. (Bown. D., Encyclopaedia of Herbs and their Uses, Dorling Kindersley, London. 1995, ISBN 0-7513-020-31.)

Anemarrhena asphodeloides is tending to grow to 0.5 m. Historically, it can be used in the treatment of ulcers. (Bown. D., Encyclopaedia of Herbs and their Uses, Dorling Kindersley, London. 1995, ISBN 0-7513-020-31.)

Platycodon grandiflorus is tending to grow to 0.5 m. Historically, it can be used in the treatment of coughs with profuse phlegm, colds, bronchitis, pleurisy, pulmonary abscesses and throat infections. (Bown. D., Encyclopaedia of Herbs and their Uses, Dorling Kindersley, London. 1995, ISBN 0-7513-020-31.)

Herbs for Sedative

According to the invention, the herb for sedative used in the composition of the invention plays the role of the Assistant herb, which assists the effect of the herb of promoting blood circulation and reducing Congealed Blood. According to the invention, the herb for sedative is selected from the group consisting of Rehmannia glutinosa or the extract thereof, Gentiana triflora or the extract thereof, Gentiann scabra or the extract thereof and Schisandra chinensis or the extract thereof. The above herbs for sedative have haemostatic and sedative effect and can reduce dizziness. The Latin name, synonyms, possible synonyms and common names of the above-mentioned herbs for sedative are shown in below Table 3:

TABLE 3

| Latin Name | Synonyms | Possible Synonyms | Common Name |
|---|---|---|---|
| Rehmannia glutinosa | Rehmannia chinensis | Digitalis glutinosa | Rehmannia, Sheng Di, |
| | | | Sheng Ti Huang, Shu Ti |
| | | | Huang, Ti Huang, Ti Huang |
| | | | Chiu |
| Gentiana triflora | | G. tubiflora | |
| Gentiana scabra | | G. scabrae | Japanese Gentian, Ling Yu, |
| | | | Lung Tan, Rindo, Ryntem |
| | | | Root |
| Schisandra chinensis | Schisandra japonica | Maximowicza chinensis | Schisandra, Tyosen-Gomisi, |
| | Maximowiczia chinensis | Sphaerostema japonica | Wu Wei Zi, Wu-wei-zi |
| | Kadsura chinensis | | |

Rehmannia glutinosa is commonly used in Chinese herbalism, where it is one of the most popular tonic herbs. Rehmannia glutinosa is tending to grow to 0.3 m. Historically, it is used to replenish energy and improves the conditions in associated with diabetes, constipation, urinary tact problems, anemia, dizziness, and regulate menstruation. Rehmannia glutinosa has effects in protecting liver, preventing the depletion of stored glycogen and inhibiting fungi such as Candida. Moreover, it can reduce glucose level, reduce blood pressure, strengthen bones and tissue and enhance fertility. Rehmannia glutinosa in combination with licorice have been used in traditional Chinese medicine for the treatment of hepatitis. In Chinese medicine theory, Rehmannia glutinosa can disperse heat from the body and alleviate night sweats and fevers and thus can be used for treating Deficient Blood patterns with the symptoms such as pallid face, dizziness, palpitations and insomnia. The root of Rehmannia glutinosa has been used in ancient times, Han dynasty, which was called Kiou and classified by prepared methods, with each preparation given a different name, i.e. Chinese foxglove, Syoujiou, Kanjiou and Zyukujiou. It is perennial growing to 0.3 m by 0.25 m and can grow in semi-shade (light woodland) or no shade. (Reid. B. E. A translation of an ancient Chinese book on edible wild foods. Fascinating. Famine Foods of the Chiu-Huang Pen-ts´ao, Taipei, Southern Materials Center 1977).

Gentiana triflora is tending to grow to 0.5 m. The Gentiana triflora contains some known compounds having good tonic for digestive system. It can be used in the treatment of jaundice, leucorrgoea, eczema, conjunctivitis, sore throat, acute infection of the urinary system, hypertension with dizziness and tinnitus. (Yeung, Him-Che, Handbook of Chinese Herbs and Formulas, Institute of Chinese Medicine, Los Angeles, 1985).

Gentiana scabra is tending to grow to 0.3 m. Gentiana scabra is used as a bitter tonic in Chinese herbalism where it promotes digestive secretions and treats a range of illness associated with the liver. It can be used in the treatment of jaundice, leucorrgoea, eczema, conjunctivitis, sore throat, acute infection of the urinary system, hypertension with dizziness and tinnitus. (Yeung, Him-Che, Handbook of Chinese Herbs and Formulas, Institute of Chinese Medicine, Los Angeles, 1985.)

Schisandra chinensis is tending to grow to 9 m. Schisandra chinensis is commonly used in Chinese herbalism, which is an excellent tonic and restorative, helping in stressful times and increasing zest for life. (Chevallier. A., The Encyclopedia of Medicinal Plants Dorling Kindersley, London 1996, ISBN 9-780751-303148.)

Herb for Flow of Qi

According to the invention, the herb for flow of qi used in the composition of the invention plays the role of Servant herb, which directs the composition of the invention to the affected region, and harmonize the herbs in the composition. According to the invention, the herb for flow of qi is selected from the group consisting of Citrus sinensis or the extract thereof, Citrus aurantium or the extract thereof, Citrus reticulate or the extract thereof, Perilla frutescens or the extract thereof, Centella asiatica or the extract thereof, Ligusticum sinense or the extract thereof, Angelica sinensis or the extract thereof, Curcuma longa or the extract thereof, Glycyrrhiza uralensis or the extract thereof, Lindera strychnifolia or the extract thereof, Gastrodia elata or the extract thereof, Saussurea costus or the extract thereof, Sparganium stoloniferum or the extract thereof, Foeniculum vulgare or the extract thereof, Angelica pubescens or the extract thereof, Arisaema amurense or the extract thereof, Acorus gramineus or the extract thereof, Anemone altaica or the extract thereof, Gentiana macrophylla or the extract thereof, Gentiana dahurica or the extract thereof and Ocimum basilicum or the extract thereof. The above herbs for flow of qi have sedative and antispasmodic effect. The Latin name, synonyms, possible synonyms and common names of the above-mentioned herbs for flow of qi are shown in below Table 4:

TABLE 4

| Latin name | Synonyms | Possible synonyms | Common names |
|---|---|---|---|
| Citrus sinensis | C. aurantium sinensis, | C. aurantium var. sinensis, | Naranja De China, Naranjo |
| | Citrus tangerina, | C. cinensis, | Dulce, Orange, Orange |
| | | C. macracantha | Blossom, Orange tree, |
| | | | Orange Doux, Portakal, |
| | | | Sweet Orange, Z'Orange |
| | | | Douce, |
| Citrus aurantium | Citrus vulgaris, | C. aurantium amara, | Bergamot, Bitter Orange, |
| | C. bigarradia, | C. aurantium ssp. aurantium, | Ch'Eng, Chih Ch'Iu, Coolie |
| | | C. bigaradia, | Orange, Hua Chu Hung, |
| | | C. vulgaris bigaradia, | Kuang Chu, Naranja Agria, |
| | | C. vulgaris var. bigaradia | Naranja De Babor, Naranjo |
| | | | Amargo, Orange, Orange |
| | | | Sure, Oranger, Seville |
| | | | Orange, Sour Orange, |
| | | | Turunc, Z'Orange Sure, |
| Citrus reticulata | | Citrus nobilis | Chu Sha Chu, Kan, |
| | | | Mandarine, Tangerine, Tran |
| | | | Bi, |
| Perilla frutescens | Perilla ocimoides | Dentidia nankinensis, | Aka Shiso, Ao Shiso, |
| | | Ocimum frutescens, | Beefsteakplant, Ji Soo, Kuei |
| | | Perilla arguta, | Jen, Nd, Pai Su, Perilla, |
| | | P. frutescens var. japonica, | Purple Perilla, Shiso, Tsu |
| | | P. nankinensis | Su, |
| | | P. ocymoides. | |
| Centella asiatica | Hydrocotyle repanda, | | Asya Sutasi, Brahmi, Daun |
| | Hydrocotyle cordifolia, | | Kaki Kuda, Gagan-gagan, |
| | Hydrocotyle asiatica | | Gotu Kola, Hydrocotyle, |
| | | | Indian Pennywort, Marsh |
| | | | Penny, Pegaga, Pegaga |
| | | | Tekukur, Pegaga Ular, |
| | | | Pegagan, Penggaga, |
| | | | Pennywort, Marsh, |
| | | | Spadeleaf, Thick-leaved |
| | | | Pennywort, White Rot, |
| Ligusticum sinense | Ligusticum chuanxiong | L. chinensis, | Chinese Privet, Kau-pau |
| | | Ligustrum indicum, | |
| | | Ligustrum microcarpum, | |
| | | Ligustrum sinense, | |
| | | Ligustrum sinense var. | |
| | | stauntonii, | |
| | | Ligustrum stauntonii, | |
| | | Ligustrum villosum, | |
| | | Phillyrea indica | |
| Curcuma longa | Curcuma aromatica, | | Indian-saffron, Turmeric, |
| | Curcuma domestica | | Yu Chiu, Curcuma, Safran |
| | | | des Indes, Gelbwurzel, |
| | | | Kurkuma, ukon, |
| | | | acafrao-da-india, Azafran |
| | | | de la india |
| Angelica sinensis | Angelica polymorpha | | Angelica, Dan Gui, Dang |
| | sinensis | | Gui, |
| Glycyrrhiza uralensis | | G. Malensis | Asian Liquorice, Chinese |
| | | | Licorice, Gan Cao, |
| | | | Gan-Cao |
| Lindera strychnifolia | | Benzoin strychnifolium, | Tendai-Uyku, Wu Yao, |
| | | Daphnidium myrrha, | |
| | | Daphnidium strychnifolium | |
| | | Dahnidium strychnifolius, | |
| | | Lindera aggregata, | |
| | | Laurus aggregata, | |
| Ocimum basilicum | | O. americanum | Ai K'Ang, Albahaca, basil, |
| | | O. basilicum var. glabratum | Basilic, Basilicon, |
| | | O. basilicum var. majus | basilicum, Chahchabram, |
| | | Ocymum basilium | Chiu Ts'Eng T'A, Feslegen, |
| | | | Hsiang Ts'Ai, I Tzu Ts'Ao, |
| | | | Lo Le, Me-Boki, Raihan Al |
| | | | Malik, Raihana, Rainan, |
| | | | Selaseh, selaseh Hitam, |
| | | | Selaseh Jantan, Selasih, |
| | | | Sweet Basil, |
| Sparganium stoloniferum | | | |
| Foeniculum vulgare | Foeniculum officinale | F. capillaceum | Adas Landi, Adas Londa, |
| | | F. foeniculum | Adas Pedas, Anis, Anis |
| | | F. vulgaris | Vert, Comino, Fenkel, |
| | | | Fennel, Finocchio Forte, |
| | | | Hinojo, Hsiao Hui Hsiang, |
| | | | Hui Shiang Chiu, Kaneer |
| | | | Razbana, L'Anis, La Nuit, |
| | | | Raziyane, Rezene, Shamar, |
| | | | Shbint, Shih Lo, Sweet |
| | | | Fennel, Tzu Mo, Tzu Mu |
| | | | Lo, Uikyo, Venkel, Wild |
| | | | Fennel, |
| Gastrodia elata | | | Ch'Ih Chien, Huan T'Ung |
| | | | Tzu, Onino-Yagara, T'Ien |
| | | | Ma, |
| Angelica pubescens | | | Du Huo |
| Arisaema amurense | | | Tian Nan Xing |
| Acorus gramineus | | Acorus calamus | Shih Ch'Ang Pu |
| Anemone altaica | | | |
| Gentiana macrophylla | | | Qin Jiao |
| Gentiana dahurica | Gentiana kurroo | | |
| | brevidens | | |
| Saussurea costus | Saussurea lappa | Aplotaxis lappa, | Costus, Kushta, Kust, Mu |
| | | Aucklandia costus | Xiang, Patchak, Quang Mu |
| | | | Xiang |

Citrus sinensis is tending to grow to 9 m. Historically, it can be used as one of sources of antioxidants and chemical exfoliants in specialized cosmetics. (Bown. D., Encyclopaedia of Herbs and their Uses, Dorling Kindersley, London. 1995, ISBN 0-7513-020-31.)

Citrus aurantium is tending to grow to 9 m. Historically, it can be used in the treatment of dyspepsia, constipation, abdominal distension, stuffy sensation in the chest, prolapse of the uterus, rectum and stomach. (Yeung Him-Che, Handbook of Chinese Herbs and Formulas, Institute of Chinese Medicine, Los Angeles, 1985.)

Citrus reticulata is tending to grow to 4.5 m. Historically, it can be used in the treatment of dyspepsia, gastrointestinal distension, coughs and profuse phlegm. (Yeung Him-Che, Handbook of Chinese Herbs and Formulas, Institute of Chinese Medicine, Los Angeles, 1985.)

Perilla frutescens is tending to grow to 0.6 m. Historically, it can be used in the treatment of asthma, colds and chills, nausea, abdominal pain, food poisoning and allergic reactions. (Bown. D., Encyclopaedia of Herbs and their Uses, Dorling Kindersley, London. 1995, ISBN 0-7513-020-31.)

Centella asiatica is tending to grow to 0.2 m. Historically, it can be used in the treatment of wounds, chronic skin conditions, venereal diseases, malaria, varicose veins, ulcers, nervous disorders and senility. (Bown. D., Encyclopaedia of Herbs and their Uses, Dorling Kindersley, London. 1995, ISBN 0-7513-020-31.)

Ligusticum sinense may be beneficial in the treatment of cerebral ischemia in humans. (Leung et al., Neurochem Res 1991 June; 16 (6): 687-92.)

Curcuma longa is a very important herb in Indian Ayurvedic medicine. Medically, it was used as a digestive aid and treatment for fever, infections, dysentery, arthritis, and jaundice and other liver problems. Traditional Chinese physicians used turmeric to treat liver and gallbladder problems, stop bleeding, and treat chest congestion and menstrual discomforts. Curcuma longa also brings attention to Commiphora (used as a hypolipidaemic agent), Picrorhiza (hepatoprotective), Bacopa (used as a brain tonic), Curcuma (antiinflammatory) and Asclepias (cardiotonic). (Jain S K, Ciba Found Symp 1994;185:153-64).

Angelica sinesis is tending to grow to 1 m, which is a well-known Chinese herb that has be used in the treatment of female ailments for thousands for years. Historically, it can be used in the treatment of a wide range of women's complaints where it regulates the menstrual cycle and relieves perios pain. (Bown. D., Encyclopaedia of Herbs and their Uses, Dorling Kindersley, London. 1995, ISBN 0-7513-020-31.)

Glycyrrhiza uralensis is tending to grow to 0.6 m. Historically, it can be used in the treatment of Addison's disease, asthma, coughs peptic ulcers, acnes, boils and sore throats. (Bown. D., Encyclopaedia of Herbs and their Uses, Dorling Kindersley, London. 1995, ISBN 0-7513-020-31.)

Lindera strychnifolia is tending to grow to 9 m. Historically, it can be used in the treatment of menstrual pain, enuresis, frequent micturation and distension with pain of the lower abdomen. (Yeung Him-Che, Handbook of Chinese Herbs and Formulas, Institute of Chinese Medicine, Los Angeles, 1985.)

Gastrodia elata is tending to grow to 1 m. Historically, it can be used in the treatment of convulsive illnesses, rheumatoid arthritis, vertigo and numbness associated with liver disharmony. (Bown. D., Encyclopaedia of Herbs and their Uses, Dorling Kindersley, London. 1995, ISBN 0-7513-020-31.)

Saussurea costus is tending to grow to 3 m. Historically, it can be used in the treatment of abdominal distension and pain, chest pain due to liver problems and jaundice, gall bladder pain, constipation associated with energy stagnation and asthma. (Bown. D., Encyclopaedia of Herbs and their Uses, Dorling Kindersley, London. 1995, ISBN 0-7513-020-31.)

Sparganium stoloniferum can be used in the treatment of abdominal pain, amenorrhoea, dysmenorrhoea and chest pain. (Yeung Him-Che, Handbook of Chinese Herbs and Formulas, Institute of Chinese Medicine, Los Angeles, 1985.)

Foeniculum vulgare is tending to grow to 1.5 m, which is commonly used household remedy. Foeniculum vulgare can be used in the treatment of a variety of complaints.

Angelica pubescens is tending to grow to 1.8 m. Historically, it can be used in the treatment of rheumatic arthritis, rheumatism, headache, toothache and abscesses.

Arisaema amurense is tending to grow to 0.5 m, which has been used in Chinese herbal medicine for thousands of years. Historically, it can be used in the treatment of cough with profuse phlegm, tumors, cervical cancer, epilepsy, tetanus and complaints involving muscular spasms. (Yeung Him-Che, Handbook of Chinese Herbs and Formulas, Institute of Chinese Medicine, Los Angeles, 1985; Bown. D., Encyclopaedia of Herbs and their Uses, Dorling Kindersley, London. 1995, ISBN 0-7513-020-31.)

Acorus gramineus is tending to grow to 0.3 m. Historically, it can be used in the treatment of digestive problems, depression and epilepsy (Yeung Him-Che, Handbook of Chinese Herbs and Formulas, Institute of Chinese Medicine, Los Angeles, 1985; Bown. D., Encyclopaedia of Herbs and their Uses, Dorling Kindersley, London. 1995, ISBN 0-7513-020-31.)

Anemone altaica is tending to grow to 0.2 m. Historically, it can be used in the treatment of poor appetite and gastro-intestinal distension, insanity, melancholia, epilepsy and impaired consciousness. (Yeung Him-Che, Handbook of Chinese Herbs and Formulas, Institute of Chinese Medicine, Los Angeles, 1985.)

Gentiana macrophylla is tending to grow to 0.6 m, which has been used in Chinese herbalism for over 2,000 years. Historically, it can be used in the treatment of digestive problems, arthritis, allergic inflammations, low-grade fever in chronic diseases, jaundice and hepatitis. (Yeung Him-Che, Handbook of Chinese Herbs and Formulas, Institute of Chinese Medicine, Los Angeles, 1985; Bown. D., Encyclopaedia of Herbs and their Uses, Dorling Kindersley, London. 1995, ISBN 0-7513-020-31.)

Gentiana dahurica is tending to grow to 0.3 m. Historically, it can be used in the treatment of arthritis, allergic inflammations, low-grade fever in chronic diseases, jaundice and hepatitis. (Yeung Him-Che, Handbook of Chinese Herbs and Formulas, Institute of Chinese Medicine, Los Angeles, 1985; Bown. D., Encyclopaedia of Herbs and their Uses, Dorling Kindersley, London. 1995, ISBN 0-7513-020-31.)

Ocimum basilicum is tending to grow to 0.45 m. Historically, it can be used in the treatment of feverish illnesses, poor digestion, nausea, abdominal cramps, gastro-enteritis, migraine, insomnia, depression and exhaustion. (Bown. D., Encyclopaedia of Herbs and their Uses, Dorling Kindersley, London. 1995, ISBN 0-7513-020-31.)

According to one preferred embodiment of the invention, the herbal composition comprises Salvia multiorhiza or Andrographis paniculata or the extract thereof, Lilium brownie or Polygala tenuifolia or the extract thereof, Rehmannia glutinosa or Schisandra chinensis or the extract thereof, and Citrus sinensis, Citrus aurantium, Citrus reticulata or Perilla frutescens or the extract thereof.

According to the present invention, the herbal composition of the invention is a mixture of four herbs at a preferred ratio of the herb for promoting blood circulation and reducing Congealed Blood:the herb for transquility:the herb for sedative:the herb for flow of qi of about 1:1:1:1 (w/w). The weight ratio may vary up to 50% per component. The “varience of the weight ratio by 50% ” means that each value of each component of the ratio may be increased or decreased by 50%. Thus, as an example, 1:1 can range from 1.5:0.5 to 0.5:1.5 (or 3:1 to 1:3).

Preparation of the Herb Composition

According to the invention, the components of the present invention can be used in the form of powder or extract extracted by conventional solvents. For the powder form, the components of the herbal composition of the present invention are air-dried and finely ground. For the extract form, the components of the present invention can be extracted by conventional solvents such as water, or any other solvent which can extract active substances from the natural substances at a temperature about 25 to the boiling point of the solvent during a period about 20 minutes to about 2 hours. The solvent from the extract solution may be removed to obtain the extract. The resulting extract can be optionally treated through filtration, concentration and dry. The exact proportion of the extracts used in the composition of this invention will depend on the concentration of active ingredients found naturally in each component. Using the guidance provided herein and a basic knowledge of drug preparation and pharmacology, one skilled in the art could easily adjust the proportions of the separate components of the composition so as to obtain a composition which has the therapeutic effects discussed herein.

According to one preferred embodiment of the invention, the extract herbal composition is produced by extracting the components with boiling, filtering the resulting extract, concentrating the filtered solution and then mixing the solution to obtain the extract composition. Preferably, the extraction is performed for 1 hour.

According to another preferred embodiment of the invention, the powder composition is produced by drying the components and grinding the dried components and then mixing the resulting components to obtain the powder composition.

Dosage Form of the Herbal Composition of the Invention

According to the invention, the herbal composition of this invention can be administered orally. The orally administered embodiments of the herbal composition of this invention can be in any conventional form such as, e.g., powder, capsules (hard or soft), tablets, elixirs, powders, granules, suspensions in water or non-aqueous media, sachets, as additives to food or beverages, or even can be made into a tea. For preparing solid orally administered compositions such as capsules or tablets, the prepared powder or extract can be mixed with a pharmaceutical carrier including conventional tableting ingredients such as corn starch, lactose, sucrose, sorbitol, talc, stearic acid, magnesium stearate, dicalcium phosphate or gums and other pharmaceutical diluents (e.g., water) to form a solid composition. Liquid preparations for oral administration may take the form of, for example, extracts, solutions, syrups or suspensions, or they may be presented as a dry product for reconstitution with water or other suitable vehicles before use. Such liquid preparations may be prepared by conventional means with pharmaceutically acceptable additives such as suspending agents, emulsifying agents, non-aqueous vehicles, preservatives and artificial or natural colors and/or sweeteners.

According to the invention, the herbal composition of this invention can be prepared as unit dosages. A unit dosage can comprise a therapeutically effective amount of each herbal extract for a single daily administration, or it can be formulated into smaller quantities of each ingredient to provide for multiple doses in a day. A unit dosage will depend upon many factors including age, size, and condition of the individual being treated and the number of times the unit will be taken in a single day. In any event, the entire daily dosage will be that which is physiologically acceptable to an individual and can be administered daily over a prolonged period of time. According to the invention, normally no more than 21 g/day of the active herb composition is administered, with part, of the total dose preferably taken at two or more different times during the day.

The present invention is also directed to methods of treating depression, comprising administering an effective amount of the herbal composition of this invention to an individual in need of depression relief. The term “effective amount” regarding the herbal composition means that amount sufficient to alleviate the symptoms associated with depression. The effective amount will depend upon the severity of the symptoms and on the responsiveness of the patient to the herbal composition. Persons of ordinary skill in the art can easily determine optimum dosages, dosing methodologies, and repetition rates.

Pharmacological Activity

According to the invention, the herbal composition can be used in relieving depressive states and anxiety. An advantage of treatment of certain cases of depression with the herbal composition of the invention is that it is rarely accompanied by side effects such as sexual dysfunction, as with many prescription antidepressants.

The pharmacological activity of the herbal composition of the invention can be evaluated by the below Forced Swimming Test (FST):

Porsolt et al. developed a Forced swimming test (FST) that includes placing a rodent (a rat or mouse) into a water tank and determining the time that the animal retain immobility. Rodents are placed individually into an transparent acrylic cuboid containing 15 cm (for the mice) or 30 cm (for the rats) of water at 24+1° C. for 6 minutes. The durations of struggling, active movement and immobility in the test is recorded by an automatic device. A 15 minutes pre-test was performed 20-24 hours prior to the test. The duration of immobility is used as a parameter of despair in the rodents. The rodents exhibiting less movement on the second FST than that on the vehicle-treated or untreated animal show that the drug has anti-depression efficacy. The FST can be used in the evaluation of a variety of potential antidepressant, including all TCAs tested (i.e., imipramine, amitryptyline, nortryptyline), MAOIs (i.e., nialamide, ipronniazid), atypical antidepressants (i.e., iprindole, mianserin, nomifensine), and ECT (Porsolt et al. Eur J Pharmacol 1978, 47:379-391; Borsini and Meli 1988, Psychopharmacology (Berl), 94:147-160.). However, the anxiolytics and phenothiazines cannot be evaluated by the FST.

The following Examples are offered by way of illustration and not by way of limitation.

### EXAMPLES

### Example 1

### Preparation of Powder Herbal Composition

The herbs Rehmannia glutinosa, Salvia miltiorrhiza, Lilium browni and Citrus Tangerina were cleaned by cold water, dried and then comminuted respectively. Each comminuted herbs was added 5 to 10 times water by weight and then extracted by boiling water for 30 to 60 minutes through extraction column, respectively. The resulting extracts were filtrated at the room temperature. The filtrated solutions were concentrated and then freezing dried, spray dried or heated with or without reducing pressure to obtain the products in power form. Each powder herbs was mixed at a ration of 1:1:1:1 to form a herbal composition of the invention.

### Example 2

### Antidepressant Efficacy of the Herbal Composition of the Invention

Animals

160-250 g of male Wistar rats were purchased from NSC (National Science Council, Taipei Taiwan). The animals were placed at constant ambient temperature (25±1° C.) with food and water available and on a 12:12-h LD cycle (lights on at 06:00, off at 18:00.)

Antipressants

Imipramine (10 mg/kg, I.P; 20 mg/kg, P.O.), and the herbal composition of the invention (6000 mg/kg, P.O. for the mice, 2400 mg/kg, P.O. for the rats) were tested respectively.

FST Test

The mice were raised in an isolated room with normal light-dark cycle, regular food and water, and continuous ultrasound condition (110 dB, 6000-7000 Hz) for 10 days. The FST test as mentioned herein was performed. The results are shown in below Table 5:

TABLE 5

| | | Mean | SD (Standard |
|---|---|---|---|
| Group | Number | (sec) | derivation) |
| Herbal composition | 5 | 114 | 28 |
| of the invention | | | |
| Imipramine | 4 | 104 | 37 |
| Vehicle | 5 | 180 | 66 |

| Test | Group | type | P values |
|---|---|---|---|
| ANOVA | Herbal composition, imipramine, vehicle | | 0.060 |
| t-test | Herbal composition vs. vehicle | | 0.035 |
| t-test | Imipramine vs. vehicle | | 0.039 |

As shown in the above Table 5, the male BALB mice administered with the imipramine shows a reduction in the duration of immobility. The herbal composition of the invention significantly reduces the duration of immobility for the male Wistar rats than the vehicle and shows the almost same effect as the imipramine.

What is claimed is:

1. A herbal composition, comprising a herb for promoting blood circulation and reducing Congealed Blood or an extract thereof, a herb for transquility or an extract thereof, a herb for sedative or an extract thereof, and a herb for the flow of qi or an extract thereof; wherein the herb for promoting blood circulation and reducing Congealed Blood is selected from the group consisting of Salvia multiorhiza, Lablab pruprueus, Dendranthema×grandiflorum, Cyperus rotundus, Prunus persica, Prunus davidiana, Paeonia suffruticosa, Senna tora, Tribulus terrestris, Paeonia lactiflora, Crocus sativus, Andrographis paniculata, Ligusticum chuanxiong, Gardenia jasminoides, and Cryptotaenia japonica, the herb for transquility is selected from the group consisting of Lilium brownii, Ziziphus jujuba, Polygala tenuifolia Atractylodes macrocephala, Triticum aestivum, Acorus calamus, Anemarrhena asphodeloides and Phyllostachys nigra, and Ocimum basilicum and the herb for sedative is selected from the group consisting of Rehmannia glutinosa, Gentiana triflora, Gentiann scabra and Schisandra chinensis and the herb for the flow of qi is selected from the group consisting of Citrus sinensis, Citrus aurantium, Citrus reticulata, Perilla frutescens, Centella asiatica, Ligusticum sinense, Angelica sinensis, Curcuma longa, Glycyrrhiza uralensis, Lindera strychnifolia, Gastrodia elata, Saussurea costus, Sparganium stoloniferum, Foeniculum vulgare, Angelica pubescens, Arisaema amurense, Acorus gramineus, Anemone altaica, Gentiana macrophylla, Gentiana dahurica.

2. The herbal composition of claim 1, wherein the herb for promoting blood circulation and reducing Congealed Blood is selected from the group consisting of Salvia multiorhiza, Andrographis paniculata and the extract thereof.

3. The herbal composition of claim 1, wherein the herb for transquility is selected from the group consisting of Lilium brownie, Polygala tenuifolia and the extract thereof.

4. The herbal composition of claim 1, wherein the herb for sedative is selected from the group consisting of Rehmannia glutinosa, Schisandra chinensis and the extract thereof.

5. The herbal composition of claim 1, wherein the herb for flow of qi is selected from the group consisting of Citrus sinensis, Citrus aurantium, Citrus reticulata, Perilla frutescens and the extract thereof.

6. The herbal composition of claim 1, wherein the herb for promoting blood circulation and reducing Congealed Blood or the extract thereof, the herb for transquility or the extract thereof, the herb for sedative or the extract thereof, and the herb for the flow of qi or the extract thereof are at a ratio ranging from about 1.5:0.5 to 0.5:1.5:1.5:0.5 to 0.5:1.5:1.5:0.5 to 0.5:1.5:1.5:0.5 to 0.5:1.5 by weight.

7. The herbal composition of claim 6, wherein the herb for promoting blood circulation and reducing Congealed Blood or the extract thereof, the herb for transquility or the extract thereof, the herb for sedative or the extract thereof, and the herb for the flow of qi or the extract thereof are at a ratio of about 1:1:1:1 by weight.

8. A herbal composition comprising Rehmannia glutinosa or the extract thereof, Salvia multiorhiza or the extract thereof, Lilium brownie or the extract thereof and Citrus sinensis or the extract thereof.

9. The herbal composition of claim 8, wherein the Rehmannia glutinosa, Salvia multiorhiza, Lilium brownie and Citrus sinensis are in the an extract form.

10. The herbal composition of claim 8, wherein the Rehmannia glutinosa or the extract thereof, Salvia multiorhiza or the extract thereof, Lilium brownie or the extract thereof and Citrus sinensis or the extract thereof are at the ratio ranging from about 1.5:0.5 to 0.5:1.5:1.5:0.5 to 0.5:1.5:1.5:0.5 to 0.5:1.5:1.5:0.5 to 0.5:1.5 by weight.

11. The herbal composition of claim 8, wherein the Rehmannia glutinosa or the extract thereof, Salvia multiorhiza or the extract thereof, Lilium brownie or the extract thereof and Citrus sinensis or the extract thereof are at the ratio of about 1:1:1:1.

12. A method of treating and/or preventing the conditions associated with depression, comprising administering to a subject in need thereof an effective amount of the composition as claimed in claim 1.

13. A method of treating and/or preventing the conditions associated with depression, comprising administering to a subject in need thereof an effective amount of the composition as claimed in claim 8.

14. The composition according to claim 1, wherein the composition is an orally administered composition in a form selected from the group consisting of capsules, tablets, elixirs, powders, granules, suspensions, sachets, food additives, beverage additives, and tea.

15. The composition according to claim 1, wherein the composition is an orally administered composition in a form selected from the group consisting of capsules, tablets, elixirs, powders, granules, suspensions, sachets, food additives, beverage additives, and tea.

16. The composition according to claim 8, wherein the composition is an orally administered composition in a form selected from the group consisting of capsules, tablets, elixirs, powders, granules, suspensions, sachets, food additives, beverage additives, and tea.
